(19) United States
(12) Patent Application Publication
(10) Pub. No.: US 2024/0115622 A1
(43) Pub. Date: Apr. 11, 2024

(54) FORMULATION FOR THE PREVENTION AND/OR TREATMENT OF COVID-19

(71) Applicant: SERVIDENCE INC., Saint-Hyacinthe (CA)

(72) Inventor: Jacques Veronneau, Saint-Hyacinthe (CA)

(21) Appl. No.: 18/482,631

(22) Filed: Oct. 6, 2023

Related U.S. Application Data
(60) Provisional application No. 63/378,769, filed on Oct. 7, 2022.

(51) Int. Cl. A61K 35/644; A61K 31/047; A61P 31/14

(52) U.S. Cl. CPC A61K 35/644; A61K 31/047; A61P 31/14

(57) ABSTRACT

A novel formulation for the prevention and treatment of COVID-19 is disclosed, featuring the antiviral properties of xylitol and bee propolis. These natural compounds were tested against porcine coronavirus (PEDV), human OC43 virus, and SARS-CoV-2. Notably, propolis and xylitol showed protective effects at concentrations below cytotoxic levels. The formulation is presented as a dental product with propolis (0.25-2.00 wt. %) and xylitol (0.25-3.0 wt. %) alongside various agents for oral and nasal applications. These findings suggest a promising approach to combat coronaviruses and provide an inventive solution for COVID-19 prevention and treatment.

### CROSS-REFERENCE TO RELATED APPLICATIONS

The present patent application claims the benefits of priority of U.S. Patent Application No. 63/378,769, entitled “Formulation for the prevention and/or treatment of covid-19”, and filed at the United States Patent and Trademark Office on Oct. 7, 2022, the content of which is incorporated herein by reference.

### FIELD OF THE INVENTION

The present invention generally relates to the field of formulations for the prevention and/or treatment of covid-19.

### BACKGROUND OF THE INVENTION

The COVID-19 pandemic has caused major repercussions on public health, causing 4,422,666 deaths and 211,288,358 cases worldwide as of Aug. 22, 2021 (World Health Organization, 2021). The causative agent of this disease is due to the severe acute respiratory syndrome coronavirus 2 (SARS-CoV-2), a coronavirus that belongs to the Betacoronavirus genus part of the Coronoviridae family of Nidovirales order. This epidemiological burden of COVID-19 is a healthcare challenge throughout the world, not only in terms of testing the limits of medical capacities, but also as an enigma considering preventive strategies and methods. Viral colonization of the nasopharyngeal niche is a dynamic and complex interplay among microflora, pathogens, host, and interference by environmental factors. Considering the mode of transmission and pathogenesis, effective oral hygiene may be beneficial to decrease the risk of transmission. In addition, gargling is deemed to bring about favorable effects through removal of oral/pharyngeal protease which helps viral replication. Currently there is not definitive cure for covid-19. Oral viral load of SARS-CoV-2 has been associated with the severity of COVID-19, and thus, a reduction in the oral viral load could be associated with a decrease in the severity of the condition. Similarly, a decrease in the oral viral load would diminish the amount of virus expelled and reduce the risk of transmission, since (i) during the first 10 days, the virus mainly accumulates at the nasal, oral, and pharyngeal area; (ii) the number of angiotensin-converting enzyme (ACE2) receptor is greater in the salivary glands as compared with the lungs; and (iii) salivary droplets represent the most relevant transmission route.

Compared with the abovementioned strains, Covid-19 is a novel virus that has high transmissibility and infectivity and poses great challenge to prevent viral transmission and infection. Human-to-human transmission occurs through indirect or direct contact during the early subclinical period with mucous membranes of the mouth, eyes, or nose. The upper respiratory tract mucosa is the first line of defense, not only as a physical barrier but also through multiple innate and adaptive immune mechanisms which are crucial for efficient antiviral.

Authors of a recent study in Seoul on covid-19 highlighted their surprising finding of clinical significance of a high viral prevalence in the saliva of infected patients: They found that viral load was the highest in the nasopharynx (patient 1=8.41 log 10 copies/mL; patient 2=7.49 log 10 copies/mL), but it was also remarkably high in the saliva (patient 1=6.63 log 10 copies/mL; patient 2=7.10 log 10 copies/mL). The authors tested a chlorhexidine mouthwash and found that the viral load in the saliva decreased transiently for 2 hours after using the mouthwash, but it increased again at 2-4 hours post-mouthwash. Still there is no covid-19 definitive cure and even less effective primary preventive strategies.

There is thus a need for a formulation usable orally or nasally for the prevention and/or treatment of covid-19.

### BRIEF DESCRIPTION OF THE DRAWINGS

FIG. 1 is showing graphics of visual scores of the CPE 3 days post infection for PEDV-infected Vero E6 cells in presence or absence of different concentrations of compounds A (Propolis), B (Xylitol), E (Remdesivir) and FBS.

FIG. 2 are illustrations of PEDV infection of VeroE6 cells in presence of FBS.

FIG. 3 are illustrations of PEDV infection of VeroE6 cells in absence of FBS.

FIG. 4 are illustrations of cytotoxicity experiment on HCT8 cells.

### DETAILED DESCRIPTION OF THE PREFERRED EMBODIMENT

A novel formulation for the prevention and/or treatment of covid-19 will be described hereinafter. Although the invention is described in terms of specific illustrative embodiment(s), it is to be understood that the embodiment(s) described herein are by way of example only and that the scope of the invention is not intended to be limited thereby.

The antiviral activities of the compounds xylitol and bee propolis, or simply propolis, were tested on animal coronavirus model virus (PEDV) and on human SARS-Co-2 virus. The antiviral effects of these natural compounds was tested on the cultured cells that are regularly used for coronavirus studies. The antiviral potentials of both compounds were tested separately and at different concentrations.

Phase 1 (months 1-4): First, the individual cytotoxicity of xylitol and propolis are to be studied. For this purpose, the compounds are added to appropriate cell cultures (Vero E6, HCT-8 and Calu-3) at different concentrations prior to inoculation of these cells with the viruses. Vero E6 cells are kidney epithelial cells derived from the African green monkey. Calu-3 cells are human lung cells. The HCT-8 cells are human ileocecal adenocarcinoma cells.

The effect of different concentrations of virus (MOI, multiplicity of infection) in the inoculum was also tested to define the effective dose of these therapeutic/protective compounds. Viruses (PEDV, OC43 and SARS-CoV-2) have been produced by infecting the corresponding cells and harvesting the supernatant when 50-60% of cell were killed by virus. The infectious titers of the viral inoculum were estimated using a standard technique of serial dilutions and evaluation of the CPE (cytopathic effect) on Vero, cells, i.e., by the Spearman-Karber method.) After inoculation, cells were monitored daily, and viral replication was assessed by microscopy (based on the CPE) and viral titration. Decrease in viral infection and viral replication indicates that there is an antiviral effect of treatment. Thus, the extent of the synergistic antiviral effect against SARS-CoV-2 in a wide dose range for both ingredients is to be analyzed.

In this first step, model coronaviruses are to be used to determine the antiviral potential of xylitol and propolis. Specifically, the porcine coronavirus (PEDV) and human OC43 virus are to be used for this purpose. These model viruses are “relatives” of the wild-type SARS-CoV-2 but working with these viruses does not require BSL3 containment. As a negative control, dilutions of the corresponding solvents in infectious media (but without virus) are used for mock-treated controls (mock-treated or mock-infected cells). Thus, the most important parameters characterizing the cytotoxicity and antiviral activity of the compounds are to be determined such as the 50% inhibitory concentration (IC50), the 50% cytotoxic concentration (CC50) as well as the dose response.

Phase 2 (months 5-6): The most important results are to be confirmed using the SARS-CoV-2 virus and performing experiments in the BSL3 facility. Since the non-cytotoxic concentrations of the propolis and xylitol have been already determined, the experiments with SARS-CoV-2 will take less time than the phase 1. The presence of cytopathic effects (CPE), TCID50 calculation or calculation of RT-qPCR Ct values are performed as described above. The data is to be analyzed using the corresponding statistical methods. In addition, the optimal doses and combinations of the therapeutic compounds are to be determined.

Potential pitfalls and mitigation plan: standard techniques and widely used kits for the evaluation of cytotoxicity (WST-1 based assay) and antiviral activity of xylitol and propolis in vitro are used. As described above, Vero E6, HCT-8 and Calu-3 cell cultures are used for this purpose. These cells are the most commonly used for in vitro studies of SARS-CoV-2. To confirm the data on a different cell culture, Caco-2 intestinal epithelial cells could be used, as SARS-CoV-2 has been reported to replicate similarly efficiently in these cells. The results are as follows:

Anticoronaviral Potential of Propolis and Xylitol

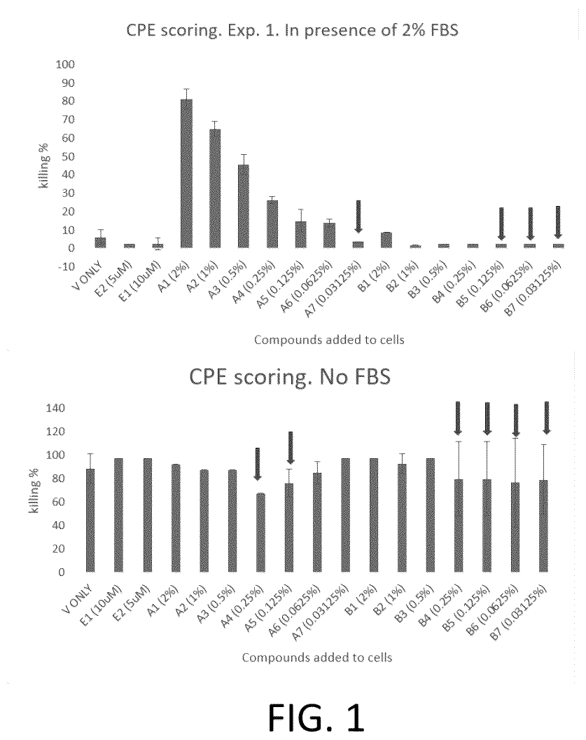

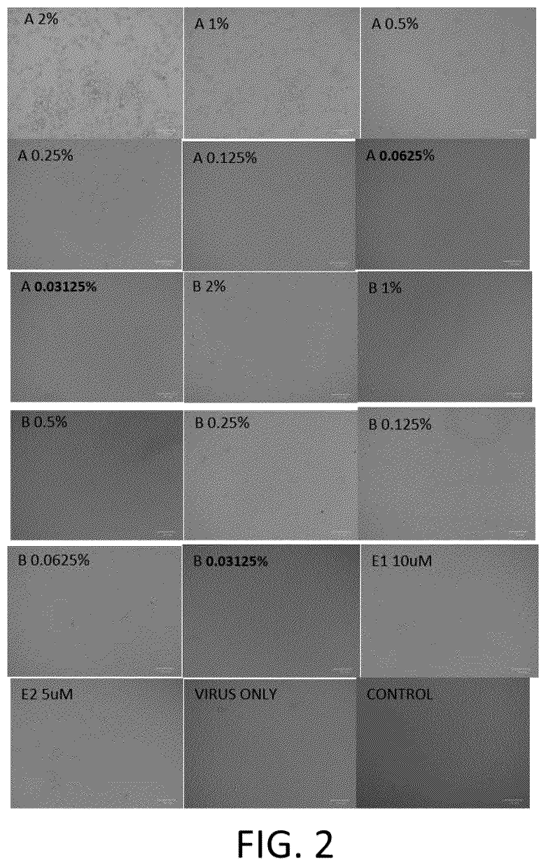

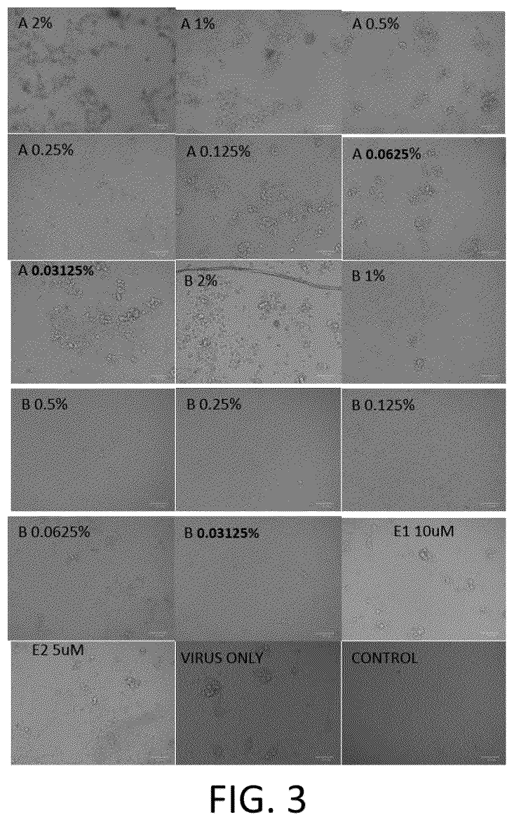

Note: Initially, it was tried to evaluate the antiviral activity of propolis and xylitol on porcine coronavirus PEDV. Showing that propolis and/or xylitol provide protection (antiviral activity) against animal and human coronaviruses would allow to make a more global conclusion about the anticoronaviral activities of these two natural products. PEDV infection in vitro requires trypsin treatment. Traditionally, in vitro infections and production of PEDV is performed in media without FBS, because FBS blocks the protease activity of trypsin and decreases or inhibits PEDV infection. Consequently, the cytotoxicity and antiviral activity of both compounds was evaluated on VERO E6 cells cultured in a medium without FBS. Unfortunately, the cytotoxicity of propolis and xylitol were very high under these conditions. Similarly, PEDV induced cytotoxicity was higher in the presence of the high concentrations of propolis and xylitol. To overcome this problem, the cytotoxicity and antiviral effects of both compounds have been assessed in a medium supplemented with 2% FBS (i.e., low concentration of FBS). The results showed that low concentration of FBS has significantly decreased the cytotoxicity of propolis and xylitol, but also presence of FBS significantly inhibited the efficiency of PEDV infection. Though it might be speculated that there is a protective effect of propolis and xylitol used at low concentrations—Propolis ≤2.0% and xylitol ≤3%—(below the cytotoxic concentrations) against PEDV infection, this finding should be better sustained. The results may be observed in FIGS. 1 to 3. In FIG. 1, visual scores of the CPE 3 days post infection for PEDV-infected Vero E6 cells in presence or absence of different concentrations of compounds A (Propolis), B (Xylitol), E (Remdesivir) and FBS. It can be noticed that at low concentrations both propolis and xylitol have shown protective effects against PEDV infection. It can be noticed that in the absence of FBS the cytotoxic effect of propolis and xylitol is high. In FIG. 2, photos of PEDV infection of VeroE6 cells in presence of FBS are shown. In FIG. 3, photos of PEDV infection of VeroE6 cells in absence of FBS are shown.

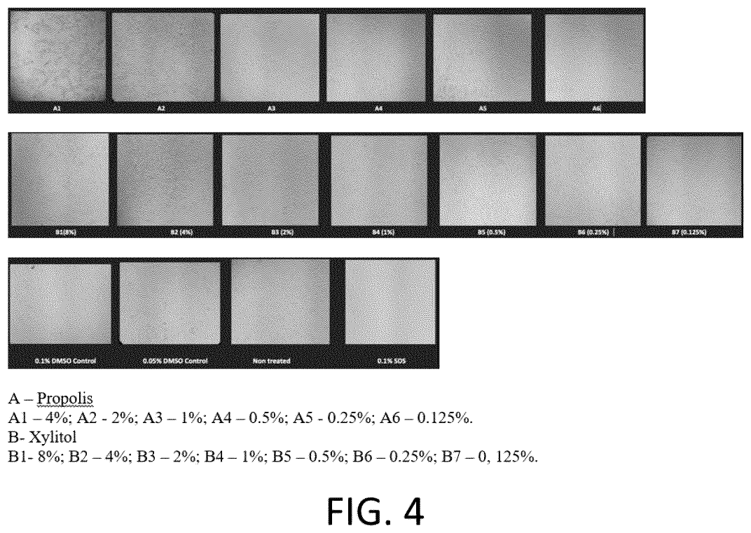

The OC43 human coronavirus production, infections and antiviral effects of propolis and xylitol against OC43 have been studied on HCT-8 and Vero E6 cells. Best results have been obtained on HCT-8 cells, therefore below data obtained on this cell line are described and shown in FIG. 4 as representative photos of cytotoxicity experiment on HCT8 cells at different concentrations of propolis and xylitol.

The experiments on HCT-8 cells infected with human respiratory coronavirus OC43 and evaluation of the propolis and xylitol antiviral effects have been repeated 4 times. OC43 is one of the most common human respiratory coronaviruses. This virus has similar overall replication strategy as human SARS-CoV-2. Like SARS-COV-3 the OC43 can cause pneumonia. Thus, it is a good model virus for studying coronaviral pathogenesis and developing antiviral strategies. The CPEs (cytotoxic effects) of two coronaviruses (OC43 and PEDV) have been measured using various methods: 1) visual scoring of the CPE; WST-1 based spectrometric OD test (results are shown); and titration of coronavirus progeny in the supernatant of the OC43-infected HCT-8 cells using Spearman-Karber method (work in progress).

In conclusion, the results showed that propolis and xylitol—with optimal effect at 0.5% concentration for propolis and for xylitol—can protect cells from coronaviral infection. Their antiviral effects depend on various factors such as cell lines, dose, presence or absence of the FBS, time of treatment, and viral species. So far, it was shown that after 2 hours of treatment exposure there was a significant effect sustained for 4 days. Based on the data, the most efficient concentrations and treatment regimen for anticoronaviral protection have been proposed. Currently, the same experimental strategy, same concentrations of propolis and xylitol are used for the SARS-CoV-2 infection experiments.

In a first embodiment of the invention, a dental formulation for the prevention and treatment of covid-19 is provided. The formulation comprising propolis and xylitol as active agents. The propolis proportion may be from about 0.25 wt. % to about 2.00 wt. % and the xylitol from 0.25 wt. % to 3.0 wt. %. The formulation may further comprise at least a carrying agent, a moisturizing agent, a thickening agent, an abrasive agent, a flavor agent, a surfactant agent and a foaming agent.

In another aspect of the invention, the formulation comprising water, barbadensis leaf juice, propolis, xylitol, hydroxyethyl cellulose, magnesium aluminum silicate, calcium carbonate, lemon flavor, sodium bicarbonate and sodium lauryl sulfoacetate.

In another aspect of the invention, the vehicle of the formulation can be oral as toothpaste or rinse and can be also nasal as a spray.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) A formulation for the prevention and treatment of covid-19, the formulation comprising propolis and xylitol as active agents.

2) The formulation of claim 1, the formulation being for oral use as toothpaste or rinse.

3) The formulation of claim 1, the formulation being for nasal use as a spray.

4) The formulation of claim 1, wherein the propolis proportion is from 0.25 wt. % to 2.00 wt. % and the xylitol proportion is from 0.25 wt. % to 3 wt. %.

5) The formulation of claim 2, wherein the propolis proportion is from 0.25 wt. % to 2.00 wt. % and the xylitol proportion is from 0.25 wt. % to 3 wt. %.

6) The formulation of claim 3, wherein the propolis proportion is from 0.25 wt. % to 2.00 wt. % and the xylitol proportion is from 0.25 wt. % to 3 wt. %.

7) The formulation of claim 1, the formulation further comprising at least one of a carrying agent, a moisturizing agent, a thickening agent, an abrasive agent, a flavor agent, a surfactant agent and a foaming agent.

8) The formulation of claim 7, the formulation comprising water, barbadensis leaf juice, hydroxyethyl cellulose, magnesium aluminum silicate, calcium carbonate, lemon flavor, sodium bicarbonate and sodium lauryl sulfoacetate.
